(19) United States
(12) Patent Application Publication
(10) Pub. No.: US 2006/0275310 A1
(43) Pub. Date: Dec. 7, 2006

(54) Method and detection and decontamination of antigens by nanoparticle-raman spectroscopy

(75) Inventors: Sulatha Dwarakanath, Austin, TX (US); John G. Bruno, San Antonio, TX (US)

Correspondence Address: Ross Spencer Garsson;Winstead Sechrest & Minick P.C., P.O. Box 50784, Dallas, TX, 78701, US

(73) Assignee: Nano Science Diagnostics, Inc., Austin, TX (US)

(21) Appl. No.: 11/222,093

(22) Filed: Sep. 8, 2005

Related U.S. Application Data
(60) Provisional application No. 60/614,668, filed on Sep. 30, 2004.

(51) Int. Cl. A61K 39/40; G01N 33/554; G01N 33/569; C07K 16/12; C07K 16/46

(52) U.S. Cl. 424/164.1; 435/7.32; 530/388.4; 977/900

(57) ABSTRACT

A composition and method of detecting antigens and killing bacteria and virus is described. The composition and method comprise a fluorescent nanoparticle conjugated to a substance capable of binding specifically to a antigen and exposing the location containing the fluorescent nanoparticle and antigen to a wavelength of light capable of exciting the fluorescent nanoparticle.

### CROSS REFERENCE

This application claims priority to and benefit of U.S. Provisional Application Ser. No. 60/614,668, filed on Sep. 30, 2004.

### RELATED PATENT APPLICATION

The following co-pending and co-assigned application contains related information and is incorporated herein by reference: U.S. Patent Application filed Dec. 3, 2004 entitled “Method and Apparatus for Low Quantity Detection of Bioparticles in Small Sample Volumes” having Srinagesh Satyanarayana as inventor.

### STATEMENT OF FEDERALLY SPONSORED RESEARCH/DEVELOPMENT

This work was funded under U.S. Army Contract No. DACA42-03-C-0063 and EPA Contract No. EP-D-04-027.

### FIELD OF THE INVENTION

The field of the invention relates generally to the detection of antigens and the killing of bacteria and virus.

### BACKGROUND OF THE INVENTION

Quantum dots are particles of matter so small that the addition or removal of an electron changes their properties. Quantum dots (QDs) have high fluorescence efficiency, lack photobleaching, and have long fluorescence (decay) lifetimes [H. Harma, T. Soukka, T. Lovgren, “Europium nanoparticles and time-resolved fluorescence for ultrasensitive detection of prostate-specific antigen,” Clin. Chem. 47 (2001) 561-568; T. Soukka, J. Paukkunen, H. Harma, S. Lonnberg, H. Lindroos, T. Lovgren, “Supersensitive time-resolved immunofluorometric assay of free prostate-specific antigen with nanoparticle label technology,” Clin. Chem. 47 (2001) 1269-1278]. These properties are allow QDs to be ultrasensitive and therefore compete with conventional fluorescent dyes for many applications.

Investigators have observed changes in QD size and emission wavelength allegedly due to oxidation and ionic strength or other environmental effects that were thought to effect the size and shape of the QD [W. G. van Sark, P. L. Frederix, A. A. Bol, H. C. Gerritsen, A. Meijerink, “Blueing, bleaching and blinking of CdSe/ZnS quantum dots,” Chemphyschem. 3 (2002) 871-879; X. Gao, W. C. Chan, S. Nie, “Quantum-dot nanocrystals for ultrasensitive biological labeling and multicolor optical encoding,” J. Biomed. Opt. 7 (2002) 532-537].

Other investigators have reported mild increases in the intensity of a peak at a lower wavelength in combination with a decrease in the peak intensity at the expected emission wavelength (blue shifts) of 30-40 nm for CdSe/ZnS QD fluorescence due to oxidation, changes in pH, the presence of divalent cations and other environmental factors [W. G. van Sark, P. L. Frederix, A. A. Bol, H. C. Gerritsen, A. Meijerink, “Blueing, bleaching and blinking of CdSe/ZnS quantum dots,” Chemphyschem. 3 (2002) 871-879].

Schaertl, et al. [S. Schaertl, F. J. Meyer-Almes, E. Lopez-Calle, A. Siemers, J. Kramer, “A novel and robust homogeneous fluorescence-based assay using nanoparticles for pharmaceutical screening and diagnostics,” J. Biomol. Screen. 5 (2000) 227-238.] reported nanoparticle immunoassay (NPIA) formats that did not require wash steps and therefore constituted true homogeneous assays for proteins and small molecular targets.

Other investigators have reported [Friedberg J. S., et al. “Antibody-targeted photolysis. Bacteriocidal effects of Sn(IV) chlorin e6-dextran-monoclonal antibody conjugates,” Ann. N.Y. Acad. Sci. 618 (1991) 383-393] and/or patented [U.S. Pat. No. 6,417,423, Koper O., Klabunde K. J., and Klabunde J. S, entitled “Reactive nanoparticles as destructive adsorbents for biological and chemical contamination,” issued Jul. 9, 2002] the toxicity of NPs toward bacteria.

There is a need for a method of detecting antigens such as bacteria, virus and proteins that does not require the removal of the unbound detection agents. Additionally, there is a need for a method to be able to kill antigens such as bacteria and virus when the detection agent binds to the antigen.

### BRIEF SUMMARY OF THE INVENTION

An embodiment of the present invention is a method of detecting bacteria comprising: (a) obtaining a fluorescent nanoparticle conjugated to a substance capable of binding specifically to a bacteria to form a conjugated fluorescent nanoparticle; (b) placing the conjugated fluorescent nanoparticle in a location where the bacteria is suspected to be; (c) exposing the location to a wavelength of light capable of exciting the conjugated fluorescent nanoparticle; (d) measuring fluorescence emission of the conjugated fluorescent nanoparticle; and (e) observing the wavelength of the measured fluorescence emission of step (d) in comparison with the wavelength of the fluorescence emission of the conjugated fluorescent nanoparticles that have not been exposed to the bacteria wherein the conjugated fluorescent nanoparticle exhibits a lower emission wavelength upon binding to the bacteria.

Another embodiment of the present invention is a method of detecting an antigen comprising: (a) obtaining a fluorescent nanoparticle conjugated to a substance capable of binding specifically to an antigen to form a conjugated fluorescent nanoparticle; (b) placing the conjugated fluorescent nanoparticle in a location where the antigen is suspected to be; (c) exposing the location to a wavelength of light capable of exciting the conjugated fluorescent nanoparticle; (d) measuring fluorescence emission of the conjugated fluorescent nanoparticle; and (e) observing the wavelength of the measured fluorescence emission of step (d) in comparison with the wavelength of the fluorescence emission of the conjugated fluorescent nanoparticles that have not been exposed to the antigen wherein the conjugated fluorescent nanoparticle exhibits a lower emission wavelength upon binding to the antigen.

Yet another embodiment of the present invention is a method of killing bacteria comprising: (a) obtaining a fluorescent nanoparticle conjugated to a substance capable of binding specifically to a bacteria to form a conjugated fluorescent nanoparticle; (b) placing the conjugated fluorescent nanoparticle in a location where the bacteria is suspected to be; and (c) binding the conjugated fluorescent nanoparticle to the bacteria, wherein the method of killing is not due to thermal activation.

Another embodiment of the present invention is a method of killing bacteria comprising: (a) obtaining a fluorescent nanoparticle comprising at least one terminal group capable of being used for conjugation; (b) placing the fluorescent nanoparticle comprising at least one terminal group capable of being used for conjugation in a location where the bacteria is suspected to be; and (c) binding the conjugated fluorescent nanoparticle to the bacteria, wherein the method of killing is not due to thermal activation.

Still another embodiment of the present invention is a method of killing bacteria comprising: (a) obtaining a fluorescent nanoparticle conjugated to a substance capable of binding specifically to a bacteria to form a conjugated fluorescent nanoparticle; (b) placing the conjugated fluorescent nanoparticle in a location where where the bacteria is suspected to be; (c) binding the conjugated fluorescent nanoparticle to the bacteria; and (d) exposing the location to microwaves.

Yet another embodiment of the present invention is a method of detecting two or more types of bacteria comprising: (a) obtaining a first fluorescent nanoparticle conjugated to a substance capable of binding specifically to a bacteria to form a first conjugated fluorescent nanoparticle, wherein the fluorescent nanoparticle conjugated to a substance capable of binding specifically to a bacteria emits at one wavelength; (b) obtaining a second fluorescent nanoparticle conjugated to a substance capable of binding specifically to a bacteria to form a second conjugated fluorescent nanoparticle, wherein the fluorescent nanoparticle conjugated to a substance capable of binding specifically to a bacteria emits at another wavelength; (c) placing the first and second conjugated fluorescent nanoparticles in a location where the bacteria is suspected to be; (d) exposing the location to a wavelength of light capable of exciting the first and second conjugated fluorescent nanoparticles; (e) measuring fluorescence emission of the first and second conjugated fluorescent nanoparticles; and (f) observing the wavelength of the measured fluorescence emission of step (e) in comparison with the wavelength of the fluorescence emission of the first and second conjugated fluorescent nanoparticles that have not been exposed to the bacteria wherein the first and second conjugated fluorescent nanoparticles exhibit lower emission wavelengths upon binding to the bacteria.

Another embodiment of the present invention is a composition for use in detection of bacteria comprising a fluorescent nanoparticle conjugated to a substance capable of binding specifically to a bacteria to form a conjugated fluorescent nanoparticle wherein the conjugated fluorescent nanoparticle exhibits a lower emission peak wavelength upon binding to the bacteria.

Yet another embodiment of the present invention is a composition for use in detection of bacteria comprising a fluorescent nanoparticle conjugated to a substance capable of binding specifically to an antigen to form a conjugated fluorescent nanoparticle wherein the conjugated fluorescent nanoparticle exhibits a lower emission peak wavelength upon binding to the antigen.

Still another embodiment of the present invention is a composition for killing bacteria comprising a fluorescent nanoparticle conjugated to a substance capable of binding specifically to a bacteria to form a conjugated fluorescent nanoparticle wherein the conjugated fluorescent nanoparticle exhibits a lower emission peak wavelength upon binding to the bacteria and the killing is not due to thermal activation.

Another embodiment of the present invention is a composition for detecting two or more types of bacteria comprising a first and second fluorescent nanoparticle conjugated to substances capable of binding specifically to the two or more types of bacteria to form a first and second conjugated fluorescent nanoparticle wherein the first and second conjugated nanoparticles emit at different wavelengths and exhibit a lower emission peak wavelength upon binding to bacteria.

Yet another embodiment of the present invention is a composition for detecting two or more types of antigen comprising a first and second fluorescent nanoparticle conjugated to substances capable of binding specifically to the two or more types of antigen to form a first and second conjugated fluorescent nanoparticles wherein the first and second conjugated nanoparticles emit at different wavelengths and exhibit a lower emission peak wavelength upon binding to the two or more types of antigen.

The foregoing has outlined rather broadly the features and technical advantages of a number of embodiments of the present invention in order that the detailed description of the present invention that follows may be better understood. Additional features and advantages of the invention will be described hereinafter.

### BRIEF DESCRIPTION OF THE DRAWINGS

The foregoing summary as well as the following detailed description of the preferred embodiment of the invention will be better understood when read in conjunction with the appended drawings. It should be understood, however, that the invention is not limited to the precise arrangements and instrumentalities shown herein. The components in the drawings are not necessarily to scale, emphasis instead being placed upon clearly illustrating the principles of the present invention. Moreover, in the drawings, like reference numerals designate corresponding parts throughout the several views.

The invention may take physical form in certain parts and arrangement of parts. For a more complete understanding of the present invention, and the advantages thereof, reference is now made to the following descriptions taken in conjunction with the accompanying drawings, in which:

FIG. 1. Diagram of Nano-Ab-Tag. A fluorescent nanoparticle 101 is bound to the antibody 103 through a molecular bridge 102.

FIG. 2. Adirondack Green NP conjugated to E. coli Ab was impregnated on a membrane 201. A serum sample 203 was added to the spot 202. If sample 203 contains E. coli, Adirondack NP conjugated to E. coli Ab will bind to E. coli. A handheld fluorometer was used to excite the serum sample containing spot 204 at 400 nm to look for the emission wavelength shift. If the sample has E. coli, then there will be a change in the intensity of the Raman Emission Peak as shown in FIG. 4.

FIG. 3. Adirondack Green NP conjugated to E. coli Ab was impregnated on two spots 202 on a membrane 201. A serum sample 203 was added to one spot 202 and a control sample 301 to the other spot 202. If a sample contains E. coli, Adirondack NP conjugated to E. coli Ab will bind to E. coli. A handheld fluorometer is used to excite the spot 202 or 204 at 400 nm to look for the change in the intensity of the Raman Emission Peak.

FIG. 4. IgM antibody-Adirondack Green EviTag (QD) fluorescence spectra of the Nano-Ab-Tag conjugates alone 401 and after binding of E. coli O111:B4 bacteria 402. There is a change in the intensity of the Raman Emission Peak associated with binding of Adirondack Green EviTag NP-labeled antibody to E. coli bacteria. The Raman Emission Peak is approximately 60 nm less than the expected emission peak (shift from 520 nm to 460 nm) and appears to occur upon binding of the NP-tagged antibody to its bacterial target. Data were obtained using a DigiLab's Model F-2500 spectrofluorometer with 400V PMT setting and 0.08 second integration time, sensitivity setting of 1 and threshold of 1. Excitation was at 400 nm with 10 nm excitation and emission slits.

FIG. 5. Fluorescence emission spectra of Fort Orange QDs before conjugation to IgG antibody 501 (panel A) and after conjugation to IgG antibody 505 (panel B). Both samples emitted in the red spectral region at 605 nm as expected when excited in the blue spectral region at 400 nm. Fluorescence emission spectra of Fort Orange QDs after binding increasing amounts of B. subtilis variant niger spores (panels C and D). When mixed with BG bacteria a large spectral shift (˜160 nm) was observed (panels C and D). Panel C depicts 10 fold dilutions of B. substilis spores. 502 is a 5×10⁶ dilution; 503 is a 5×10⁵ dilution and 504 is a 5×10⁴ dilution of B. subtilis spores. Panel D depicts the fluorescence emission spectra 506 with use of the highest concentration of B. substilis spores tested in this experiment, 1 mg/ml. Data were obtained using DigiLab's Model F-2500 spectrofluorometer with 400V PMT setting and 0.08 second integration time, sensitivity setting of 1 and threshold of 1. Excitation was at 400 nm with 10 nm excitation and emission slits.

FIG. 6. Combined fluorescence spectra (showing the excitation peak at 400 nm+20 nm and emission spectra out to 700 nm) for Fort Orange QD-anti-Salmonella IgG antibody with increasing amounts of heat killed S. typhimurium bacteria. The emission peak of Fort Orange QD-anti-Salmonella IgG antibody bound to S. typhimurium is around 460 nm as opposed to an expected emission peak for Fort Orange QD-anti-Salmonella IgG of around 600 nm. Line 603 is 5 CFU; 602 is 5×10² CFU and 601 is 5×10⁴ CFU of S. typhimurium bacteria.

FIG. 7. Combined fluorescence emission spectra for Fort Orange QD-anti-LPS O111:B4 DNA aptamers with increasing amounts of live E. coli O111:B4. Excitation was at 400 nm+20 nm. An increase in the intensity of the Raman Emission Peak approximately 140 nm away (from 600 nm to 460 nm) appears to occur upon binding of the NP-tagged antibody to its bacterial target. This may be referred to as a blue shift or downshift. Data were obtained using DigiLab's Model F-2500 spectrofluorometer with 400V PMT setting and 0.08 second integration time, sensitivity setting of 1 and threshold of 1. Excitation was at 400 nm with 10 nm excitation and emission slits. Line 702 is Fort Orange NPs alone, 701 is a ten-fold dilution, 703 is a hundred-fold dilution, 704 is a thousand-fold dilution, 705 is a ten thousand-fold dilution of live E. coli O111:B4.

FIG. 8. Panel A is a brightfield image of E. coli O111:B4 stained with anti-E. coli IgM antibody-Adirondack Green QD conjugate. Panel B shows the same sample under fluorescence microscopy using a fluorescein filter cube (blue excitation). Panel C is a brightfield image of E. coli O111:B4 stained with anti-E. coli IgM antibody-Fort Orange QD conjugate and panel D is a blue-excited fluorescence image of the same sample. All images were taken at a total magnification of 400×.

FIG. 9. Panels A and B are photographs of the plates from Experiment 2 (Example 15). Panel A—before counting of colonies; Panel B—after counting with blue marks from a permanent marker to show where the colonies were. The amount of EviTag Amine NPs (ETA), EviTag Carboxyl NPs (ETC) or IgM-EviTag Carboxyl (IgM-ETC) added is indicated in μg. Note that the IgM-Adirondack Green ETC and Adirondack Green ETC alone groups were toxic to the bacteria even at 10 ug of added reagents and exhibited increased toxicity at 20 ug and 40 ug of added reagents. All plates received 50 μL of the 10⁻⁴ bacterial dilution. Panel C depicts the number of bacterial CFUs remaining following exposure to NPs or NPs conjugated to E. coli specific antibodies at various volumes of NPs or NPs conjugated to E. coli specific antibodies. Type 1 NPs are NPs with amine side chains and Type 2 NPs are NPs with carboxyl side chains. Bar 901 provides the results after the addition of buffer; Bar 902 addition of 10 ug of NP Type 1; Bar 903 addition of 20 ug of NP Type 1; Bar 904 addition of 40 ug of NP Type 1; Bar 905 addition of buffer; Bar 906 addition of 10 ug of NP Type 1 conjugated to E. coli specific Ab; Bar 907 addition of 20 ug of NP Type 1 conjugated to E. coli specific Ab; Bar 908 addition of 40 ug of NP Type 1 conjugated to E. coli specific Ab; Bar 909 addition of buffer; Bar 910 addition of 10 ug of NP Type 2; Bar 911 addition of 20 ug of NP Type 2; Bar 912 addition of 40 ug of NP Type 2; Bar 913 addition of buffer; Bar 914 addition of 10 ug of NP Type 2 conjugated to E. coli specific Ab; Bar 915 addition of 20 ug of NP Type 2 conjugated to E. coli specific Ab and Bar 916 addition of 40 ug of NP Type 2 conjugated to E. coli specific Ab.

FIG. 10. Schematic diagram of the detection scheme for biological warfare agents using an antibody conjugated to NPs. A spray 1009 containing nanoparticles 1002 conjugated to antibodies 1003 is applied from a container 1007 to the wall 1001. The expanded view 1008 depicts a nanoparticle 1002 conjugated to antibody 1003 and a nanoparticle 1002 conjugated to antibody 1003 bound to antigen 1004 and 1005. A fluorescent light source 1010 is provided to the area of spray 1008 by a handheld fluorometer 1006 and the emission wavelength is detected by the handheld fluorometer 1006.

FIG. 11. An embodiment of the invention was performed with heat killed 0157:H7 strain of E. coli and Fort Orange NPs from Evident Technologies, N.Y. The experiment was performed as in FIGS. 4-6. Line 1101 indicates the results following the addition of 3×10⁶ CFU, 1102 the addition of 3×10⁴ CFU, 1103 the addition of 3×10² CFU and 1104 the addition of 3 CFU of heat killed 0157:H7 E. coli. There is a change in the intensity of the fluorescence emission of the “Raman Bio-Peak™.” at about 460 nm.

FIG. 12. Another embodiment of the invention was performed with heat killed 0157:H7 strain of E. coli and QDs from Quantum Dot Corp., CA. The experiment was performed as in FIGS. 4-6. Line 1201 indicates the results following the addition of 3×10⁶ CFU, 1202 the addition of 3×10⁴ CFU, 1203 the addition of 3×10² CFU and 1204 the addition of 3 CFU of heat killed 0157:H7 E. coli. QDs alone emit at 565 nm. There is a change in the intensity of the fluorescence emission of the “Raman Bio-Peak™” at about 460 nm.

### DEFINITIONS

An “antibody” is an immunoglobulin molecule that only interacts with the antigen that induced its synthesis in cells of the lymphoid series, or with an antigen closely related to it.

An “antigen” is a substance capable of inducing synthesis of an antibody and being bound by such antibody. This substance is selected from the group including but not limited to bacteria, virus, viral particles and protein.

“Aptamers” are specific RNA or DNA oligonucleotides or proteins which can adopt various three dimensional configurations. Because of this aptamers can be produced to bind tightly to a specific molecular target.

“Bacteria” are one cell organisms.

“Blue shift” is an increase in a peak of a lower wavelength combined with a decrease in intensity of the peak at the expected emission wavelength.

“CFU” are colony forming units.

“Fluorescence” is the emission of light of one wavelength upon absorbtion of light of another wavelength.

“Log kill” is the amount of reduction in the number of bacteria or virus. A ten fold reduction in the number of bacteria or virus is equal to 1 log kill.

“Quantum dots” are particles of matter so small that the addition or removal of an electron changes their properties.

“Raman Bio-Peak™ emission” is the increase in intensity of the Raman Emission Peak that corresponds with the number of bound bacteria.

“Raman Emission Peak” is the peak at about 460 nm wavelength for water.

“Wavelength” is the distance between two waves of energy.

### DETAILED DESCRIPTION OF THE INVENTION

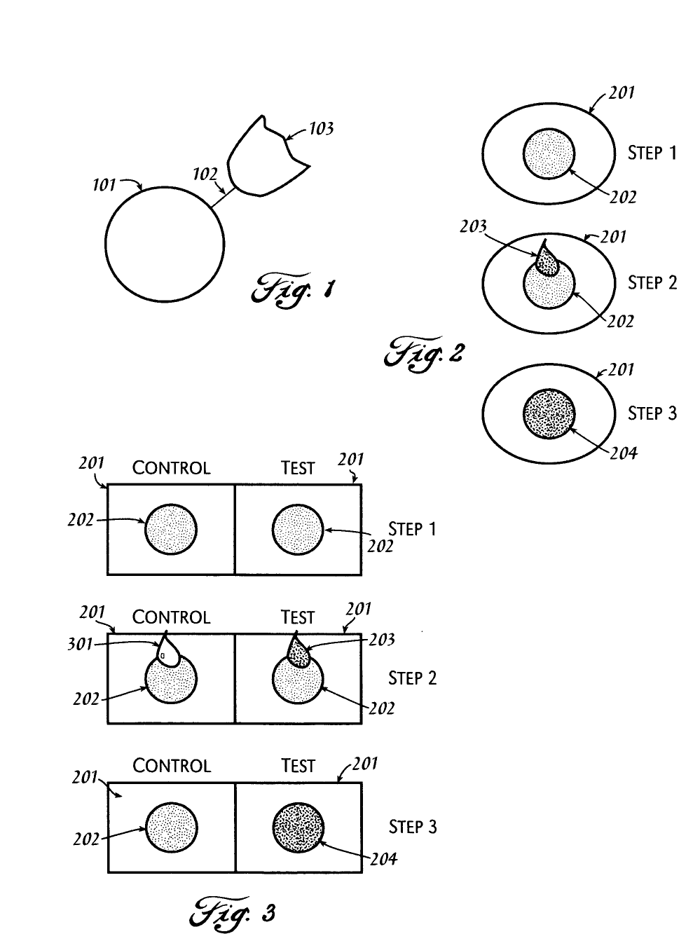

CdSe/ZnS quantum dots (QDs) exhibit change in the Raman Emission Peak when conjugated to antibodies or DNA aptamers that are bound to bacteria. A Nano-Ab-Tag can be formed (FIG. 1). The intensity of the Raman Emission Peak was found to increase with the number of bound bacteria, which is a very minor component of the natural fluorescence spectrum of these QDs. This emission has been named the “Raman Bio-Peak™ emission.”

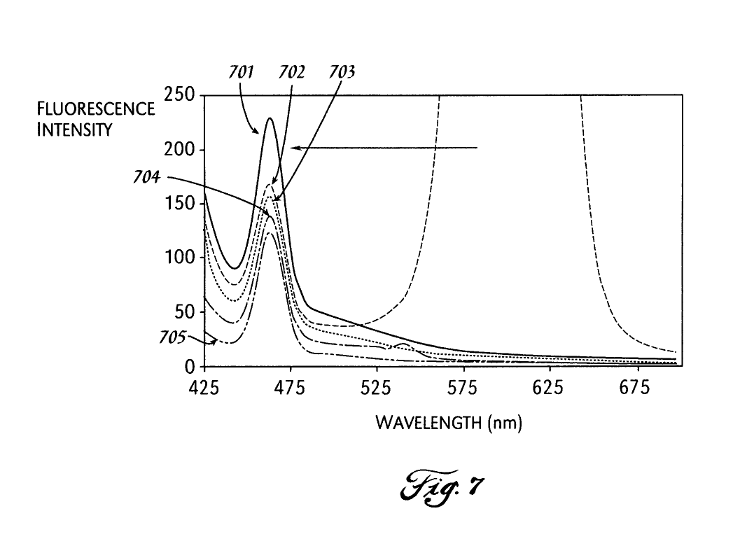

The change appears to occur by adding energy from the fluorescence emission to a minor peak near 440-460 nm that exists for the unconjugated and unbound QDs (FIGS. 5-7). This minor peak near 440-460 nm appears to increase in intensity with the concentration of analytes in the systems studied with various species of bacteria as the target analytes. Other QD compositions besides CdSe/ZnS may exhibit similar shifts.

QDs essentially “confine” electrons (and their resulting photons) to a particular spatial dimension. Therefore, the size of the QD generally dictates fluorescence emission wavelength. By inference then, if the size of the QD was to change during a binding reaction and become smaller, the fluorescence emission wavelength might at a lower wavelength than its expected emission wavelength.

QD-antibody or aptamer conjugates that bind bacterial or other cell surfaces may experience a different chemical interface, which may alter the QDs' size or deform their shape, thereby altering their emission wavelength. This hypothesis has been tested on several bacterial-antibody-QD and aptamer-QD systems.

This appears to be the first report of a Raman Emission Peak change (increase in a peak of a lower wavelength combined with a decrease in intensity of the peak at the expected emission wavelength, also known as blue shift) due to binding of receptor-QD conjugates (whether antibodies or aptamers) to bacterial surfaces. This change can be used in the detection and determination of the number of antigens, such as bacteria, present.

The shift of the fluorescence emission peak to a lower wavelength may be due to environmental factors such as differences in hydrophobicity, hydrophilicity, pH, electric charge, etc. The shift might also be due to physical deformation of the QDs when the QDs near the surface of the bacteria. Since QDs are quantum confined “boxes” for electrons, when the size or shape of the “box” changes, the confined wavelength and emission wavelength may also change. Thus, if a spherical QD were to become compressed (ovoid) near the bacterial surface upon antibody binding by even a nanometer or less, it could dramatically influence the emission wavelength. The wavelength shift may be due to changes in the chemical environment of the QD conjugates when they encounter the bacterial surface and may be due to physical deformation of the QD that changes the quantum confinement state. Regardless of the mechanism, these changes in the “Raman Bio-Peak™ emission” at about 460 nm suggest their suitability for use in homogeneous (one step) assays using QD-receptor conjugates without wash steps.

The nanoparticles being used are biologically inert, conjugation ready, nano-scale particles. They are based on the unique characteristics of nanocrystal quantum dots, including, but not limited to, those composed of CdSe/ZnS and Metal Oxide NPs. They offer the optical and chemical characteristics, ideal for high stability, color multiplexing, single excitation assays, and they are available with carboxyl or amine terminal groups for conjugation, and in sizes ranging from 30 to 50 nanometers and have multiple reactive functional groups per particle. “Adirondack Green” and “Fort Orange” nanoparticles both have excitation maxima near 400 nm and emission peaks of 520 nm and 600 nm, respectively. The Metal Oxide NPs have Europium Em=600 nm and Turbium Em=564 nm.

Antibody (150 kD IgG or 900 kD IgM)-QD conjugates and smaller 18 kD (60 base) DNA aptamer-QD conjugates exhibit dramatic changes in fluorescence emission peaks of at least 140 nm upon binding to the bacterial surface. Both the Adirondack Green and Fort Orange QD-conjugates exhibited a “Raman Bio-Peak™ emission” in the vicinity of 440 nm to 465 nm. The 440-460 nm peak is barely present in fluorescence spectra of either kind of QD without chemical conjugation or binding to bacteria. Both types of QDs are composed of CdSe/ZnS, but differ in average core diameter (4.3 and 6.3 nm respectively for Adirondack Green and Fort Orange). Therefore, these two types of QDs might be expected to share some fluorescence spectral features such as minor secondary emission peaks. The intensity (energy distribution) of this natural secondary fluorescence peak appears to grow significantly upon binding of the QD conjugates to bacteria in several different receptor (antibody or aptamer) and bacterial (B. subtilis, E. coli, or Salmonella) assay systems. This observation may make QD systems potentially very valuable for immunoassays, molecular biology applications and biological warfare agent detection.

NPs can be conjugated to specific antibodies and used to sensitively detect antigens by both fluorescence microscopy and spectrofluorometry. A fluorescence surface scanner can be used without the need for wash steps to eliminate background fluorescence because the emission peak for the unbound NPs is at a different wavelength. The method of detection can be completed in a variety of time frames including as little as 15, 10, 5 or 2 minutes and can detect the presence of equal to or greater than 20, 10 or 3 bacteria or viral particles or as little as 15 μg or even 5 μg of protein. The method is capable of detecting within 3 colony forming units of the actual number of bacteria.

NPs are somewhat toxic to bacteria, but this toxicity is greatly enhanced by the binding of antibody-NPs to the surface of target bacteria, making an antibody-NP decontamination and detection spray feasible. This observed toxicity is not due to thermal activation.

Results Indicate:

1. Fluorescent NPs (termed as semiconductor NPs), composed of CdSe/ZnS from Evident Technologies can be conjugated to antibodies and used to sensitively detect antigens, including, but not limited to, bacteria, virus and proteins, as illustrated by the assay dilution curves.

2. Detection may be extended from test tubes or microscope slides to other surfaces, of a substantial and consistent change in the “Raman Bio-Peak™ emission” when antibody-NPs bind to target bacteria. All systems shifted to approximately 460 nm, which was a minor emission peak for the antibody-Adirondack Green NP conjugate alone. Quantitative results can be obtained in less than 10 minutes from spot test and on surfaces.

3. Fluorescent NPs showed some toxicity (i.e., decreased colony counts) compared to controls, but antibody-NPs were more toxic or deadly to target bacteria, even at lower concentrations. It appears that no one has previously reported on the increased toxicity of the antibody-NP conjugate. This lethality can be exploited in targeted therapy for human and veterinary uses for reducing infections and inactivating cancer cells.

This is an improvement over existing rapid tests because of increased sensitivity and quantitation of the results in less than 15 minutes.

A variation is to use quantum confined nanosize particles that fluoresce and can be conjugated to an antibody or nucleic acid. For instance, nanoparticles, either semiconductor or metal oxide with a lanthanide core, can be conjugated to an antibody or nucleic acid, through a chemical linkage. Nano Crystals technology produces a metal oxide nanoparticle. [U.S. Pat. Nos. 5,422,489; 5,422,907; 5,446,286; 5,455,489; 5,637,258; 5,952,665; 6,036,886; 6,300,640; 6,361,824 and 6,452,184]

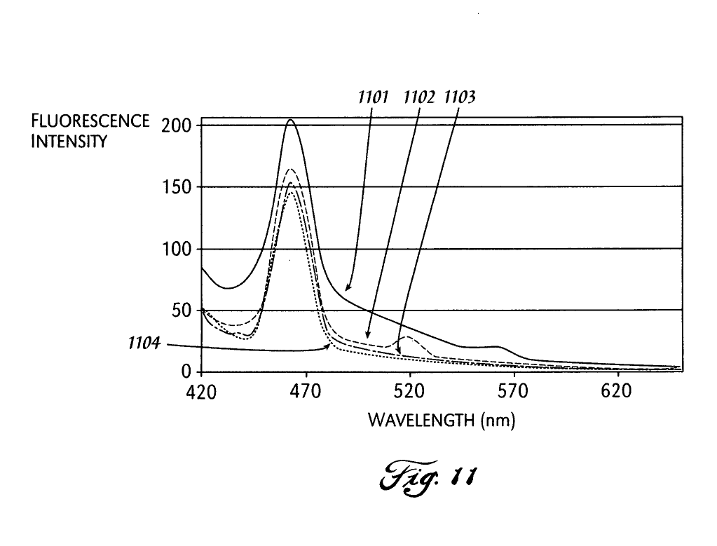

A means of detecting bacteria on surfaces, such as walls and floors, is by the use of an aerosol, that could be sprayed into the surfaces where the antibodies would bind to the bacteria, as shown in FIG. 10. The detection systems used in such an aerosol are based on innovations that capitalize on the ability of antibody nanoparticle conjugates to change the intensities of their optical emission wavelengths upon binding to bacteria.

### EXAMPLES

The following examples are provided to more fully illustrate some of the embodiments of the present invention. It should be appreciated by those of skill in the art that the techniques disclosed in the examples which follow represent techniques discovered by the inventors to function well in the practice of the invention, and thus can be considered to constitute exemplary modes for its practice. However, those of skill in the art should, in light of the present disclosure, appreciate that many changes can be made in the specific embodiments that are disclosed and still obtain a like or similar result without departing from the spirit and scope of the invention.

### Example 1

### QDs, Antibodies, Aptamers, and Bacteria

“Adirondack Green” and “Fort Orange EviTags™” QDs were purchased from Evident Technologies Inc. (Troy, N.Y.). These classes of QDs have excitation maxima near 400 nm and emission peaks of 520 nm and 600 nm, respectively. Both amine and carboxyl derivatives of these QDs were used on separate occasions and conjugated to antibodies or aptamers as described below. Murine anti-Escherichia coli O111:B4 monoclonal IgM was purchased from Novus Biologicals, Inc. (Littleton, Colo.). Goat anti-Salmonella (CSA-1) polyclonal IgG was purchased from Kirkegaard Perry Laboratories (KPL; Gaithersburg, Md.). Rabbit anti-Bacillus subtilis variant niger polyclonal antiserum was the kind gift of Dr. Richard Karalus at the Calspan Research Center in Buffalo, N.Y. Round 5 (60 base) DNA aptamers were generated by the magnetic bead (MB)-based method of Bruno and Kiel [J. G. Bruno, J. L. Kiel, “Use of magnetic beads in selection and detection of biotoxin aptamers by ECL and enzymatic methods,” BioTechniques. 32 (2002) 178-183] using lipopolysaccharide (LPS) O111:B4-conjugated MBs. The LPS O111:B4 was obtained from Sigma-Aldrich (St. Louis, Mo.) and was conjugated to Dynal, Inc. (Lake Success, N.Y.) M-270 amine-MBs using sodium periodate and cyanoborohydride chemistry as recommended by Dynal, Inc. Live E. coli O111:B4 were obtained from American Type Culture Collection (ATCC; Rockville, Md.). Heat-killed S. typhimurium was obtained from KPL and B. subtilis var niger spores were obtained from the U.S. Army's Dugway Proving Ground, Utah.

A simulant for anthrax, known as Bacillus globigii (BG) and its antibody was utilized in these experiments. BG spores were obtained from Dugway Proving Ground, while BG polyclonal antiserum was the kind gift of Dr. Richard Karalus at the Calspan Research Center in Buffalo, N.Y.

### Example 2

### QD Conjugation to Antibodies

In general, 6.25 μg (25 μL) of EviTag QDs (amino or carboxyl terminated) were added to 4.3 mL of sterile deionized water and 0.5 mL of sterile 10×PBS (0.1 M phosphate buffered saline, pH 7.2 to 7.4) with 200 μl (10 mg) of sterile EDC (1-ethyl-3-(3-dimethylamino propyl)carbodiimide). The solution was allowed to incubate at room temperature (RT) for 1 h with occasional mixing. Then 0.2 mg of IgM or 1 mg of IgG was added and the solution was further incubated at RT for 2 h. The reaction was stopped with 5.5 ml of sterile 1 M Tris (pH 7.4). The liquid was transferred to a spin filter apparatus (Omega Macrosep 300k by Pall Corp., Ann Arbor, Mich.) and spun at 3,000×G for 30 to 60 min. The retentate containing the antibody-QD conjugate was stored at 4° C. until used.

### Example 3

### QD Conjugation to Aptamers

Twenty-five μl (approximately 12.5 μg) of round 5 anti-LPS O111:B4 DNA aptamers that were modified by incorporation of 5′-disulfide primers during the final PCR amplification were added to 450 μl of PBS. The disulfide ends were reduced to sulfhydryls by addition of 10 μl of dithiothreitol to 200 μl of the disulfide-end modified aptamers in 800 μl of tris-borate-EDTA (TBE) buffer for 15 min at RT with mixing. The 5′-sulfhydryl-aptamers were desalted on a Pharmacia PD-10 column (Sephadex G-25) that had been equilibrated with 1×PBS. One ml fractions were collected from the column and the peak fractions were pooled based on absorbance readings at 260 nm. Twenty-five μl of 200 mM N-β-maleimidopropionic acid (BMPA; Pierce Biotechnology Corp., Rockford, Ill.) was added and the solution was incubated for 2 h at RT. The material was transferred to Omega Nanosep 3000 MWCO spin columns (Pall Corporation, Ann Arbor, Mich.) and centrifuged for 15 min at 5,000×G to remove excess BMPA. Seventy μl of semi-purified BMPA-aptamers were added to 270 μl of nuclease-free sterile deionized water with 50 μl of 10×PBS, 100 μl of amine-EviTags™ (Adirondack Green or Fort Orange QDs) plus 10 μl of 100 mg/ml ethylene diaminecarbodiimide (EDC). The mixture was incubated at RT for 2 h and 500 μl of 1 M Tris-HCl (pH 7.4) was added to quench the reaction. Unconjugated aptamers were removed by two spins through an Omega Nanosep column 100,000 MWCO (Pall Corporation, Ann Arbor, Mich.) at 12,000×G for 5 min initially and again for 10 min. The retentate was washed on the spin column in 500 μl of 1× (0.01M) PBS by centrifugation at 12,000×G for 5 min. The retentate pellet was resuspended in 1 mL of sterile 1×PBS and stored at 4° C.

### Example 4

### Bacterial Tube Assays Using Antibody-QD and Aptamer-QD Conjugates

Three types of bacterial immunoassays were examined. In the first assay, a bolus of freshly cultured E. coli O111:B4 was scraped from the surface of a tryptic soy agar plate and suspended in 5 ml of 1×PBS. The cells were dispersed ten times with a 5 ml syringe and 20 Gauge needle by vigorous suction and ejection. This produced a single bacterial cell suspension of approximately 2.8×10⁶ cells/ml. Ten-fold dilutions were made in 1×PBS from this stock. In the second and third assays, ten-fold dilutions of heat-killed S. typhimurium (1 mg/ml stock concentration) and B. subtilis var niger spores (1 mg/ml stock) were made in 1×PBS. Fifty μl of antibody-QD conjugates (approximately 10 μg of IgM or 50 μg of IgG) were added per tube and allowed to react for 1 h at RT with slow mixing. Bacteria were pelleted by centrifugation and washed three times by resuspension and centrifugation in 1.5 mL of 1×PBS. The only differences for aptamer-QD tube assays were that approximately 10 μg of 95° C.-heated (single-stranded) DNA aptamer-QDs were used and dilutions were made in aptamer binding buffer (1×BB; [J. G. Bruno, J. L. Kiel, “Use of magnetic beads in selection and detection of biotoxin aptamers by ECL and enzymatic methods,” BioTechniques. 32 (2002) 178-183]). The aptamer-QD assay was only attempted for E. coli O111:B4. Controls consisted of bacteria without antibody-QD or aptamer-QD addition.

### Example 5

### Assays Using Antibody-QD Complexes to Detect Proteins

A protein to be detected will be suspended in 5 ml of 1×PBS. Ten-fold dilutions will be made in 1×PBS from this stock. Fifty μl of antibody-QD conjugates (approximately 10 μg of IgM or 50 μg of IgG) will be added per tube and allowed to react for 1 h at RT with slow mixing. The proteins will be pelleted by centrifugation and washed three times by resuspension and centrifugation in 1.5 mL of 1×PBS. It is predicted that as low as 5 μg of protein, could be detected.

### Example 6

### Assays Using Antibody-QD Complexes to Detect Virus

A virus to be detected will be suspended in 5 ml of 1×PBS. Ten-fold dilutions will be made in 1×PBS from this stock. Fifty μl of antibody-QD conjugates (approximately 10 μg of IgM or 50 μg of IgG) will be added per tube and allowed to react for 1 h at RT with slow mixing. The virus will be pelleted by centrifugation and washed three times by resuspension and centrifugation in 1.5 mL of 1×PBS. It is predicted that as few as 3 virus could be detected.

### Example 7

### Spectrofluorometry

Samples were diluted up to 4 ml in 1×PBS or IX BB as appropriate and analyzed in plastic cuvettes on a DigiLab's (Randolph, Mass.) Model F-2500 spectrofluorometer with 400 V PMT setting, 0.08 second integration time, and sensitivity and threshold settings of 1. Excitation was always set at 400 nm with 10 nm excitation and emission slits. Bacteria were carefully resuspended immediately prior to acquisition of emission spectra.

### Example 8

### Fluorescence Microscopy

To confirm that QD-antibody conjugates were bound to bacteria, 100 μl of undiluted antibody-Adirondack Green and Fort Orange QD conjugates were added to heat-fixed bacterial smears on microscope slides. Antibody-QD conjugates were allowed to bind for 1 h at RT and were then rinsed with 1×PBS for several minutes. Coverslips were added to 1×PBS-wetted slides and slides were examined on an Olympus BH-2 fluorescence microscope with a standard fluorescein filter cube (blue excitation for both the Adirondack Green and Fort Orange QDs) and photographed or digitally captured with a video camera.

### Example 9

### Fluorescence Emission Change in the Intensities of the “Raman Bio-Peak™”

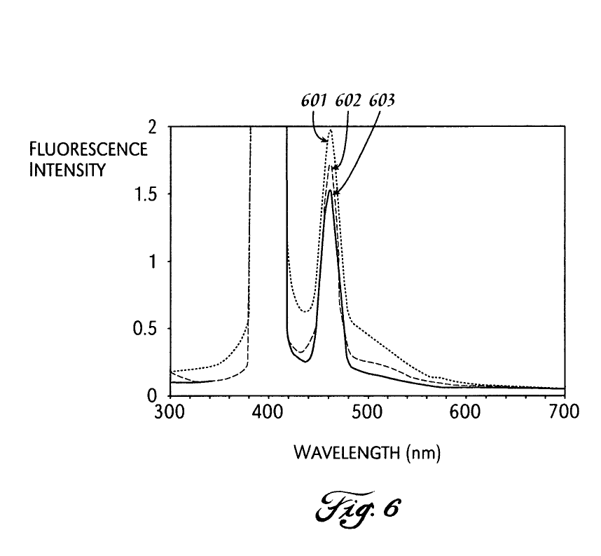

Adirondack Green-labeled anti-E. coli O111:B4 IgM antibodies were allowed to bind a 1:10 dilution of the stock E. coli O111:B4 bacteria. In FIG. 4, IgM Adirondack Gree EviTag (QD) fluorescence spectra are shown for the Nano-Ab-Tag conjugates alone 401 and after binding of E. coli 0111:B4 bacteria 402. This dilution probably represents approximately 2.8 million bacteria per ml. Also noted from FIG. 4 is a minor secondary peak for the Adirondack Green at about 460 nm. This natural secondary emission peak is seen throughout the data and may be a common minor peak for CdSe/ZnS materials, which resides around 440 nm to 460 nm, but grows in intensity (i.e., gains energy) upon binding of the antibody or aptamer-QD to bacterial surfaces. The change in the “Raman Bio-Peak™” is shown FIGS. 5-7.

FIGS. 5-7 indicate that there is at least a semi-quantitative nature to the intensity of the secondary peaks. Increasing concentrations of bacteria increased the intensity of the secondary emission peak, while the fluorescence intensity of the primary peaks (at 520 or 600 nm) either diminished or disappeared. This can be referred to as a blue shift or downshift. This observation suggests energy transfer of the QDs from their primary to their secondary emission states. This energy transfer is especially noticeable in FIG. 5 (panels B and D). In panel D, the highest concentration of B. subtilis spores (1 mg/ml) was added to the antibody-QD system and the secondary peak grew from slightly less than 6 units in relative intensity (FIG. 5, panel B) to just under 600 units (FIG. 5, panel D), while the primary peak intensity at 600 nm shrunk (not visible in FIG. 5).

As reflected in FIGS. 5-7, it appears that it does not matter if the receptor was an antibody or an aptamer. The semi-quantitative increase in intensity of the secondary peak at the expense of the primary peak as a function of bacterial concentration is again witnessed in FIGS. 5-7.

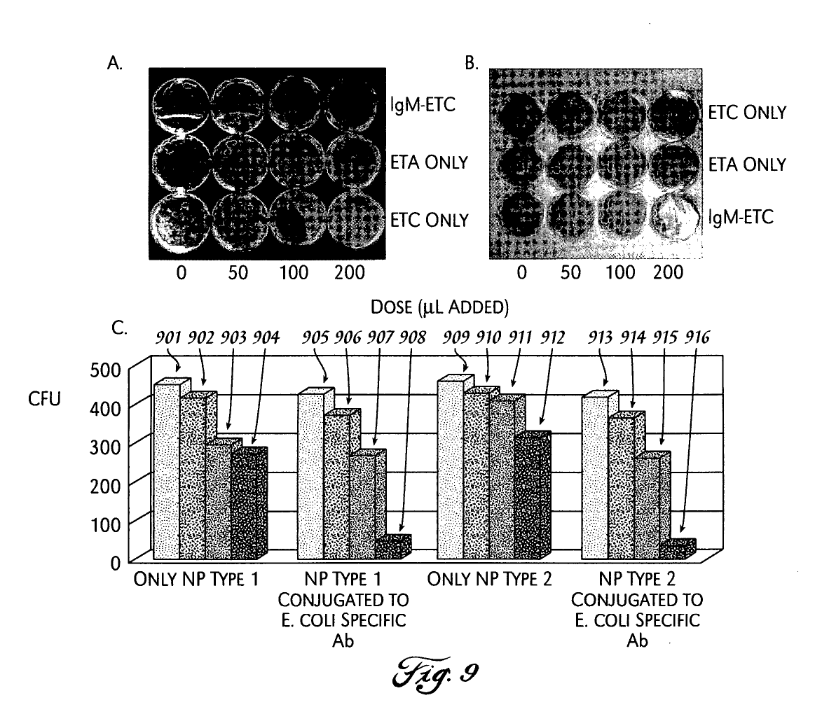

To confirm that antibody-QD conjugates were binding to the bacteria, fluorescence microscopy was employed and demonstrated well-defined, punctate fluorescence of the antibody-QDs on E. coli for both the Adirondack Green- and Fort Orange-antibody systems, as shown in FIG. 8. The fluorescence images do not seem to show a noticeable color shift.

### Example 10

### Application of Nano-Ab Tags: Observations of Fluorescence Change in “Raman Bio-Peak™ Emission” Upon Binding of Antibody-NP Conjugates to Bacteria and its Importance in Diagnostics

The following work on Nano-Ab tags was done with Nanoparticles from Evident Technologies. Fluorescence emission wavelength shifts upon binding of NP-tagged antibodies or aptamers to their bacterial targets enables the development of a fluorescence assay that would allow a user to perform antibody-NP reaction with an antigen and then scan the reaction surface at a specified wavelength to detect presence of the antigen without wash steps to eliminate fluorescence background.

The wavelength shift in has been tested with E. coli, Salmonella and Bacillus globigii (Anthrax simulant) to show that the shift occurs in different bacteriological systems.

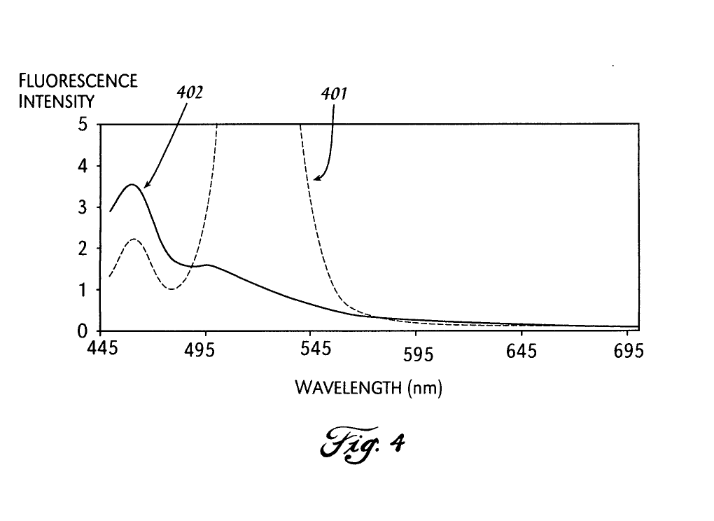

Adirondack Green NP conjugated to E. coli Ab was impregnated on a membrane. A serum sample was added to the spot. If sample contains E. coli, Adirondack NP conjugated to E. coli Ab will bind to E. coli. (FIG. 2). A handheld fluorometer was used to excite the spot at 400 nm to look for the emission wavelength shift. If the sample has E. coli, then the emission spectra will show a change in “Raman Bio-Peak™ emission.” If there is no E. coli in the sample there will be no change.

A spot testing is possible in 2 minutes. The washing step/steps for the unbound NP-Ab from the mixture has been eliminated. Also, the fluorometer can capture the intensity of the emission and using calibration algorithm allowing quantitative information to be obtained from the same test.

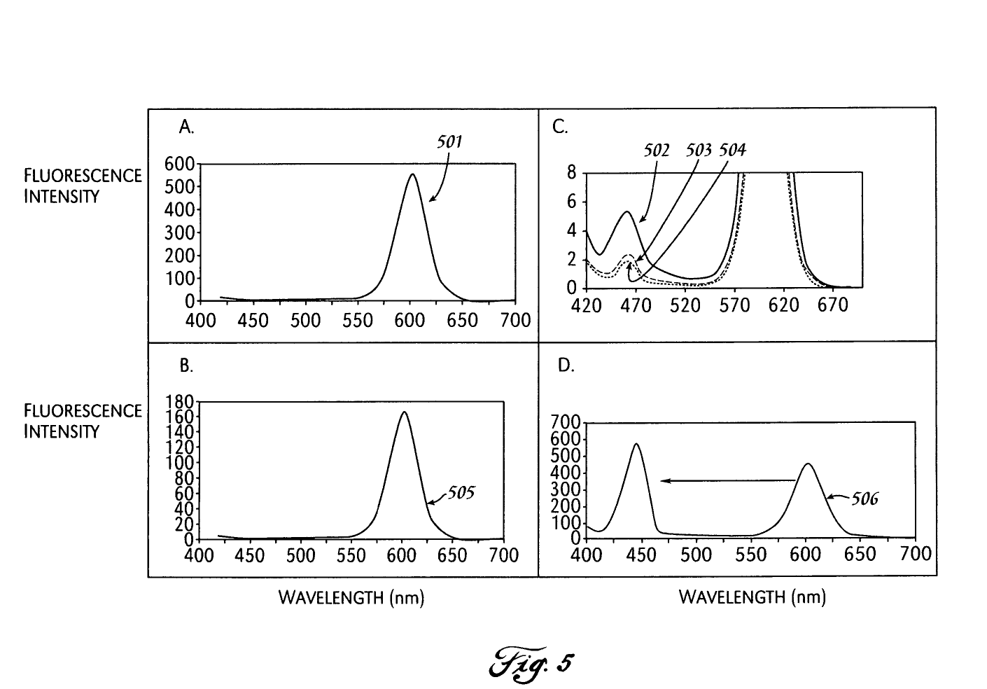

Adirondack Green NP conjugated to E. coli Ab was impregnated on two spots on a membrane. A serum sample was added to one spot and a control sample to the other spot. If a sample contains E. coli, Adirondack NP conjugated to E. coli Ab will bind to E. coli (FIG. 3). A handheld fluorometer was used to excite the spot at 400 nm to look for the emission wavelength shift. If the sample has E. coli, then the emission spectra will change in “Raman Bio-Peak™ emission” as shown in FIG. 4. If there is no E. coli in the sample there will be no change.

### Example 11

### Fort Orange NPs and BG System

An EviTag-NP assay system using polyclonal IgG anti-Bacillus globigii antiserum (from CUBRC, Buffalo, N.Y.) and Bacillus globigii (BG) bacteria obtained from Dugway Proving Ground was investigated. This second assay system was investigated for two major reasons: (1) BG is a simulant for Bacillus anthracis (Anthrax) (2) the inventors wanted to verify the emission change with a different EviTag NP system, especially in the Anthrax simulant. Fort Orange NPs from Evident Technologies were used for making the NP-Ab tags. Fort Orange is a Red emitter expected to emit at about 600 nm. FIG. 5 Panel A shows the emission of the NP alone peaking at 605 nm. Panel B shows the emission of NP with BG Ab peaking at 605 nm. Panel C in FIG. 5 shows an increase in emission at a lower wavelength than expected when the EviTag NP-BG Ab was mixed with BG (1 mg/ml). The emission peak starts at about 417 nm and peaks at about 447 nm. The same experiment was performed with Lake Placid Blue NPs and BG antibodies with similar results. Lake Placid Blue NPs have Ex=400 nm and Em=490 nm.

### Example 12

### Fort Orange Evitag NPs and Salmonella System

An EviTag-NP assay system using polyclonal IgG anti-Salmonella antiserum and heat-killed Salmonella typhimurium bacteria obtained from Kirkegaard Perry Laboratories, Inc. (KPL; Gaithersburg, Md.) was investigated. Fort Orange Evitag NPs were used for two major reasons: (1) the inventors wanted to verify the emission wavelength shift with a bacterial system, and (2) the numbers of heat-killed bacteria are known with some degree of accuracy from KPL (i.e., 5×10⁹/mL stock), thus making semi-quantitative assays possible. FIG. 6 shows a similar, yet more pronounced blue wavelength emission change for a Fort Orange EviTag-anti-Salmonella antibody system that was expected to emit at about 600 nm, the emission wavelength of the Fort Orange EviTag NPs and their antibody conjugate emit. The emission was shifted to a lower wavelength by approximately 140 nm (FIG. 6) when bound to the heat-killed Salmonella bacteria.

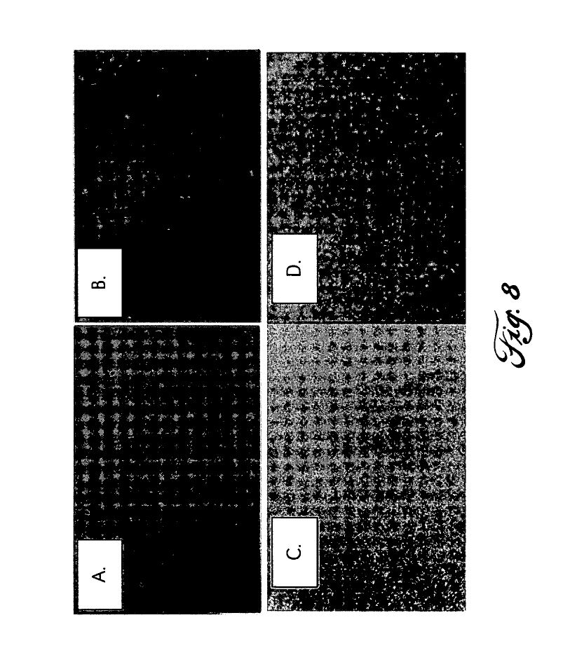

In FIGS. 4, 5 and 6, the NPs were suspended and reacted in PBS buffer. The data for FIG. 7 was obtained in a Aptamer Binding buffer, which has a high concentration of salt. The peak at 464 nm for the NP (Fort Orange) is attributed to the effect of salt. The high concentration of salt in Aptamer Binding Buffer makes the NPs Cluster more (FIG. 7) compared to PBS buffer (FIG. 6). Also, the clustering is seen when the antibody (FIG. 5) or aptamer (FIG. 7 Apt FO-NPs) binds to NP. Then the shift is dramatic once the bacteria binds as seen in FIGS. 4 (panel B), 5 (panel D) and 6.

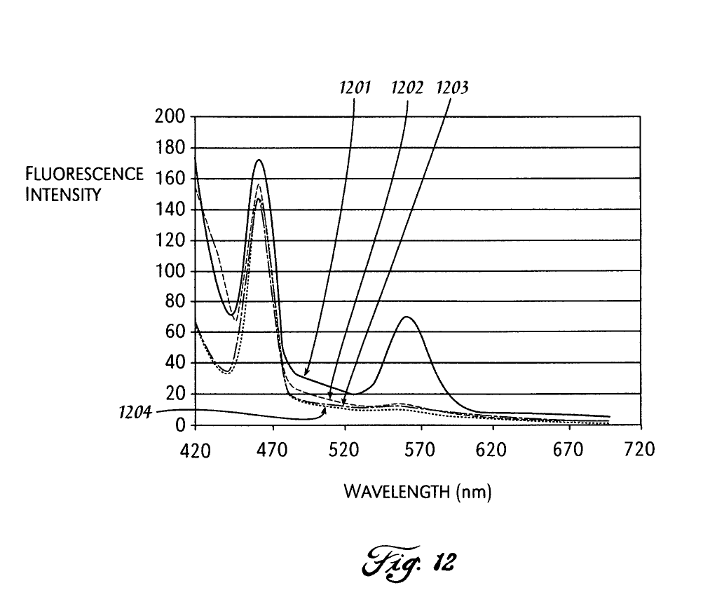

In FIGS. 11 and 12, the experiment for detection was repeated with heat killed 0157:H7 strain of E. coli with 2 different types of nanoparticles. The protocol of the experiment was the same as for FIGS. 4-6. FIG. 11 shows the results for nanoparticles from Evident Technologies, N.Y. and FIG. 12 shows the results for Quantum Dot Corp., CA. Both show a change in the fluorescence emission at about 460 nm.

### Example 13

### Therapeutic Uses of Nano-Ab Tags

Antibody conjugates have higher level of lethality for E. coli bacteria compared to nanoparticles alone. In Experiment 1, four different treatments were examined: (1) unexposed control (UC) E. coli O111 bacteria, (2) microwave-exposed controls (EC), (3) microwave-exposed plus IgM-EviTag Amine NPs (E-IgM-ETA), and (4) microwave-exposed plus IgM-EviTag Carboxyl (E-IgM-ETC). In the first experiment, 150 μL of the IgM conjugates were used or substituted by 150 μL of additional phosphate buffered saline (PBS, 0.1M and pH 7.2). A stock suspension of live E. coli O111 was made by taking a loopful of live bacteria off a Tryptic Soy Agar (TSA) culture plate that had been in an incubator and making a single cell suspension by pipetting in 10 mL of sterile room temperature PBS until no lumps were seen.

Fifty μL of the stock suspension was added to each of four sterile 12×75 mm polypropylene tubes with 150 μL of IgM-ETA or IgM-ETC or PBS as appropriate for that particular treatment group. The tubes were mixed thoroughly before drawing any samples or transfers so as to avoid settling errors. Tubes were allowed to react at room temperature for 20 minutes. Thereafter, dilutions were made from 10⁻¹ to 10⁻⁷ in 1 mL volumes of sterile PBS in sterile polypropylene 12×75 mm tubes. Then 200 μL of each dilution was plated on TSA plates and spread by means of sterile plastic cell spreaders (“hockey sticks”) to ensure uniform distribution of bacterial cells across the plate surfaces.

Culture plates from the microwave-exposed groups were placed one by one into the center of a conventional microwave oven (Emerson Model MW8618CB) for 30 second exposures at the “low” power setting. As an indicator of energy deposition, TSA plate surface temperatures were taken for each group after exposure. The unexposed control surface temperatures, reflect the starting temperatures of all the groups prior to microwave exposure. These temperature data are presented in Table 1. Temperature data were acquired with a handheld IR laser thermometer in scan mode.

TABLE 1
Experiment 2: TSA Plate Surface Temps (degrees F.)

| Dilution | UC | EC | U-Ab-ETA | E-Ab-ETC |
|---|---|---|---|---|
| 1.00E−04 | 74(78) | 131(156) | 76(77) | 82(104) |
| 1.00E−05 | 75(78) | 79(90) | 75(78) | 90(153) |
| 1.00E−06 | 77(78) | 82(102) | 75(76) | 83(109) |
| 1.00E−07 | 81(81) | 82(109) | 76(80) | 84(112) |

The first value in each data set in Table 1 is the temperature from the center of the plate and the value in parentheses is the highest temperature seen on the plate in scan mode immediately upon taking the plate from the microwave oven. Plates were rotated on the circular glass platform in the microwave oven to aid in making microwave exposure uniform for the 30 second exposure period. After exposure, the plates were collected together and cultured in an incubator at 35° C. overnight (17 hours). Plate counts were then acquired and recorded as in Table 2.

### Example 14

### Therapeutic Uses of Nano-Ab Tags

The results of the experiment are given in Tables 1, 2 and 3. Table 1 gives the IR measured surface temperatures of each plate (i.e., center and highest recorded). Tables 2 and 3 consist of colony counts taken at 17 hours after incubation and at 41 hours. The re-incubation was attempted to see if undetected colonies would emerge over time.

TABLE 2
Experiment 2: Colony Counts (17 Hours of Culture)

| Dilution | UC | EC | U-Ab-ETA | E-Ab-ETC |
|---|---|---|---|---|
| 1.00E−04 | 138 | 0 (melted) | 1 | 1 |
| 1.00E−05 | 6 | 26 | 0 | 0 |
| 1.00E−06 | 1 | 2 | 0 | 0 |
| 1.00E−07 | 0 | 0 | 0 | 0 |

U-Ab-ETA = Unexposed IgM-EviTag Amine;
E-Ab-ETA = Exposed IgM-EviTag Amine

TABLE 3
Experiment 2: Colony Recounts (41 Hrs of Culture)

| Dilution | UC | EC | U-Ab-ETA | E-Ab-ETC |
|---|---|---|---|---|
| 1.00E−04 | 305 | 0 | 2 | 12 |
| 1.00E−05 | 39 | 60 | 0 | 2 |
| 1.00E−06 | 6 | 3 | 0 | 2 |
| 1.00E−07 | 0 | 0 | 0 | 0 |

The EC 10⁻⁴ dilution plate was overexposed to the microwave field for 60 seconds and clearly killed all the bacteria, because the plate reached a peak temperature of 156° F., which even melted the agar temporarily. Hence, clearly microwaves alone are effective against bacteria, if sufficient energy is deposited on target. However, this may not be acceptable in all building decontamination scenarios or in the human body. The solution is to focus the microwave energy enabling use of lower power with equal lethality.

The effect of IgM-NPs alone (in the absence of microwaves) on E. coli was examined. Interestingly, IgM-ETA particles without microwaves appear to be highly toxic to E. coli as demonstrated in both Tables 2 and 3. The toxicity of antibody-NP conjugates and NPs alone in the absence of microwaves as seen in Experiment 2 was tested.

### Example 15

### Therapeutic Uses of Nano-Ab Tags

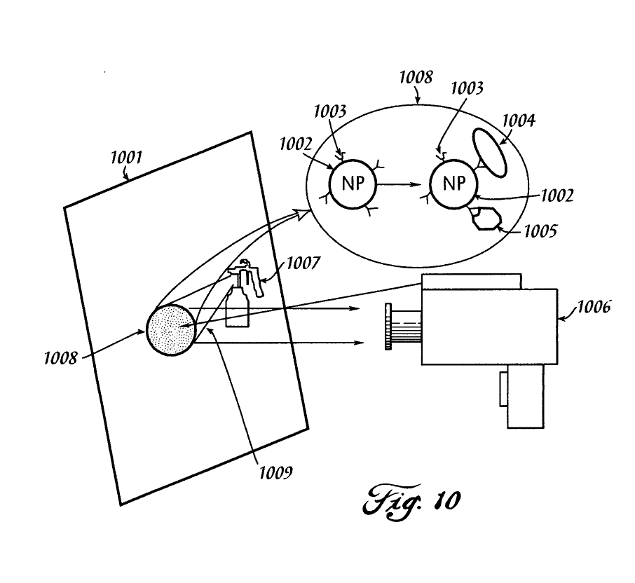

In Experiment 2, it was confirmed that E. coli O111 specific IgM-EviTag NP conjugates can be highly toxic to E. coli O111. In addition, it appears that the ETC NPs alone (without antibody conjugation or microwave augmentation) were also significant toxicants for E. coli, but to a lesser degree than the antibody-NP conjugates. In the case of the ETC toxicity, the inventors hypothesized that this may be due to the carboxyl linker reacting with the E. coli surface to bring the NP in close contact or proximity with the bacteria, much like the IgM antibody does. By contrast, the ETA NPs were much less toxic (Table 4 and FIG. 9). A 10⁻⁴ dilution was used for all the plates in this experiment and no microwave energy was applied, however, the level of NPs and IgM-ETC were varied from 0 to 40 μg as shown and reactions occurred for 20 minutes at RT. A volume of 250 μL was added to each plate and spread with a sterile spreader. Toxicity can be measured as log kill. Use of 40 μg of NP Type 1 conjugated to an E. coli specific Ab or NP Type 2 conjugated to an E. coli specific Ab provided a log kill of 0.885 or 1.14 respectively (FIG. 9). Use of 40 μg of NP Type 1 or NP Type 2 provided a log kill of 0.275 or 0.146 respectively (FIG. 9). Additional colonies did emerge in all the plates after the initial overnight incubation at 35° C. when plates were left at RT for three days. But, the more toxic treatment groups continued to have significantly less colonies than the other plates. The IgM-NPs showed a concentration-dependent ability to kill the bacteria when 40 μg of conjugate were added (last line of Table 4).

TABLE 4

| | |
|---|---|
| Amount | Results of Experiment 2 (Second Trial) |

| Added (μg) | ETA | IgM-ETA | ETC | IgM-ETC |
|---|---|---|---|---|
| 0 | TNTC | TNTC | TNTC | TNTC |
| 10 | TNTC | TNTC | TNTC | TNTC |
| 20 | TNTC | TNTC | TNTC | TNTC |
| 40 | TNTC | 48 | TNTC | 31 |

TNTC = “Too Numerous to Count” or greater than 300 colony forming units (CFUs).
All results are given in CFUs.
ETA = EviTage Amine NPs Only.
ETC = EviTag Carboxyl NPs only.
IgM = ETA or IgM-ETC = IgM conjugates.

Table 4 shows that without any microwave treatment, the NP-conjugates were rendered lethal to bacteria.

The lethality of the NP conjugates could be useful in many medical applications including but not limited to: (1) targeted lethality of cancer cells (specific antibodies for a particular type of cancer can target the NP to the particular site and upon shining of UV light render the cancer cells lethal) and (2) an anti infectious agent for human and veterinary uses.

This technology can be further extended to a different type of NPs comprised of metal oxides with a built in impurity from lanthanides (from Nano Crystals Technology). The advantages of these particles are that they have very sharp bands of emission, thus avoiding false positives in the system.

In all cases investigated here, the magnitude of the wavelength change in “Raman Bio-Peak™ emission” appears to be specific to the nanoparticle used as a taggant, and not to the antibody with which it is conjugated. This allows the capability of using different nanoparticles to distinguish among various bacterial agents that may be present simultaneously.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above and below described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

It is therefore, contemplated that the claims will cover any such modifications or embodiments that fall within the true scope of the invention.

### REFERENCES

All publications and patent applications mentioned in this specification are indicative of the levels of those skilled in the art to which this invention pertains. All publications and patent applications are herein incorporated by reference to the same extent as if each individual publication or patent application was specifically and individually indicated to be incorporated by reference.
Bruno J. G. and Kiel J. L., “Effect of radio-frequency radiation (RFR) and Diazoluminomelanin (DALM) on the growth potential of bacill,” Electricity and Magnetism in Biology and Medicine, pp. 231-233 (1993) San Francisco Press.
Bruno J. G. and Kiel J. L., “In vitro selection of DNA aptamers to anthrax spores with electrochemiluminescence detection,” Biosensors & Bioelectronics 14:457-464 (1999).
Bruno J. G. and Kiel J. L., “Use of magnetic beads in selection and detection of biotoxin aptamers by ECL and enzymatic methods,” BioTechniques 32:178-183 (2002).
Bruno J. G. and Mayo M. W., “A simple color image analysis method for assessment of germination based on differential fluorescence staining of bacterial spores and vegetative cells by acridine orange,” Biotechnic. & Histochem. 70:175-184 (1995).
Bruno J. G., Ulvick S. J., Uzzell G. L., Tabb J. S., Valdes E. R., and Batt C. A., “Novel immuno-FRET assay method for Bacillus spores and E. coli O157:H7,” Biochem. Biophys. Res. Comm. 287:875-880 (2001).
Bruno, J. G. and Yu H., “Sensitive bacterial pathogen detection using an immunomagnetic—electrochemiluminescence instrument: Potential military and food industry applications,” Biomed. Products 20:20-22 (1995).
Friedberg J. S., et al., “Antibody-targeted photolysis. Bacteriocidal effects of Sn(IV) chlorin e6-dextran-monoclonal antibody conjugates,” Ann. N.Y. Acad. Sci. 618:383-393 (1991).
Gao X., Chan W. C., Nie S., “Quantum-dot nanocrystals for ultrasensitive biological labeling and multicolor optical encoding,” J. Biomed. Opt. 7:532-537 (2002).
Harma H., Soukka T., Lovgren T., “Europium nanoparticles and time-resolved fluorescence for ultrasensitive detection of prostate-specific antigen,” Clin. Chem. 47: 561-568 (2001).
Hirsch L. R., et al., “Nanoshell-mediated near-infrared thermal therapy of tumors under magnetic resonance guidance,” Proc. Natl. Acad. Sci., USA. 100:13549-13554 (2003).
Jaiswal J. K., et al., “Long-term multiple color imaging of live cells using quantum dot bioconjugates,” Nat. Biotechnol. 21:47-51 (2003).
Kirschvink J. L. “Microwave absorption by magnetite: a possible mechanism for coupling nonthermal levels of radiation to biological systems,” Bioelectromagnetics. 17:187-194 (1996).
Kloepfer J. A., et al. “Quantum dots as strain and metabolism-specific microbiological labels,” Applied and Environmental Microbiology, 69:4205-4213 (2003).
Pinnick RG., Hill S. C., et al. and Bruno, J. G., “Fluorescence particle counter for detecting airborne bacteria and other biological particles,” Aerosol Sci. Technol. 23:653-664 (1995).
Schaertl S., Meyer-Almes F. J., Lopez-Calle E., Siemers A., Kramer J., “A novel and robust homogeneous fluorescence-based assay using nanoparticles for pharmaceutical screening and diagnostics,” J. Biomol. Screen. 5: 227-238 (2000).
Soukka T., Paukkunen J., Harma H., Lonnberg S., Lindroos H., Lovgren T., “Supersensitive time-resolved immunofluorometric assay of free prostate-specific antigen with nanoparticle label technology,” Clin. Chem. 47:1269-1278 (2001).
U.S. Pat. No. 6,303,316, Kiel J. L., Bruno J. G., Parker J. E., Alls J. L., Batishko C. R., and Holwitt E. A., entitled “Organic semiconductor recognition complex and system,” issued Oct. 16, 2001.
U.S. Pat. No. 6,417,423, Koper O., Klabunde K. J., and Klabunde J. S., entitled “Reactive nanoparticles as destructive adsorbents for biological and chemical contamination,” issued Jul. 9, 2002.
U.S. Pat. No. 6,569,630, Vivekananda J. and Kiel J. L., entitled “Method and compositions for aptamers against anthrax,” issued May 27, 2003.
Van Sark W. G., Frederix P. L., Bol A. A., Gerritsen H. C., Meijerink A., “Blueing, bleaching and blinking of CdSe/ZnS quantum dots,” Chemphyschem. 3:871-879 (2002).
Yu H. and Bruno J. G., “Immunomagnetic-electrochemiluminescent detection of Escherichia coli 0157 and Salmonella typhimurium in foods and environmental water samples,” Appl. Environ. Microbiol. 62:587-592 (1996).

What is claimed is:

1. A method of detecting bacteria comprising:
(a) obtaining a fluorescent nanoparticle conjugated to a substance capable of binding specifically to a bacteria to form a conjugated fluorescent nanoparticle;
(b) placing the conjugated fluorescent nanoparticle in a location where the bacteria is suspected to be;
(c) exposing the location to a wavelength of light capable of exciting the conjugated fluorescent nanoparticle;
(d) measuring fluorescence emission of the conjugated fluorescent nanoparticle; and
(e) observing the wavelength of the measured fluorescence emission of step (d) in comparison with the wavelength of the fluorescence emission of the conjugated fluorescent nanoparticles that have not been exposed to the bacteria wherein the conjugated fluorescent nanoparticle exhibits a lower emission wavelength upon binding to the bacteria.

2. The method of claim 1, wherein the substance capable of binding specifically to a bacteria is an antibody.

3. The method of claim 1, wherein the substance capable of binding specifically to a bacteria is an aptamer.

4. The method of claim 1, wherein the fluorescent nanoparticle comprises cadmium selenide/zinc sulfate.

5. The method of claim 1, wherein the fluorescent nanoparticle comprises a quantum confined nanosize particle.

6. The method of claim 1, wherein the fluorescent nanoparticle is a metal oxide with a lanthanide core.

7. The method of claim 1, wherein the method of detecting bacteria is quantitative.

8. The method of claim 1, wherein the method of detecting bacteria occurs in at most about 15 minutes.

9. The method of claim 1, wherein the method of detecting bacteria occurs in at most about 10 minutes.

10. The method of claim 1, wherein the method of detecting bacteria occurs in at most about 5 minutes.

11. The method of claim 1, wherein the method of detecting bacteria occurs in at least about 2 minutes.

12. The method of claim 1, wherein the method of detecting bacteria can detect the presence of at least about 20 bacteria.

13. The method of claim 1, wherein the method of detecting bacteria can detect the presence of at least about 10 bacteria.

14. The method of claim 1, wherein the method of detecting bacteria can detect the presence of at least about 3 bacteria.

15. The method of claim 1, wherein the method of detecting bacteria can detect within about 5 colony forming units of the actual number of bacteria.

16. The method of claim 1, wherein the method of detecting bacteria can detect within about 3 colony forming units of the actual number of bacteria.

17. A method of detecting an antigen comprising:
(a) obtaining a fluorescent nanoparticle conjugated to a substance capable of binding specifically to an antigen to form a conjugated fluorescent nanoparticle;
(b) placing the conjugated fluorescent nanoparticle in a location where the antigen is suspected to be;
(c) exposing the location to a wavelength of light capable of exciting the conjugated fluorescent nanoparticle;
(d) measuring fluorescence emission of the conjugated fluorescent nanoparticle; and
(e) observing the wavelength of the measured fluorescence emission of step (d) in comparison with the wavelength of the fluorescence emission of the conjugated fluorescent nanoparticles that have not been exposed to the antigen wherein the conjugated fluorescent nanoparticle exhibits a lower emission wavelength upon binding to the antigen.

18. The method of claim 17, wherein the method of detecting an antigen occurs in at most about 15 minutes.

19. The method of claim 17, wherein the method of detecting an antigen occurs in at most about 10 minutes.

20. The method of claim 17, wherein the method of detecting an antigen occurs in at most about 5 minutes.

21. The method of claim 17, wherein the method of detecting an antigen occurs in at least about 2 minutes.

22. The method of claim 17, wherein the antigen is a viral particle.

23. The method of claim 22, wherein the method of detecting the viral particle can detect the presence of at least about 20 viral particles.

24. The method of claim 22, wherein the method of detecting the viral particle can detect the presence of at least about 10 viral particles.

25. The method of claim 22, wherein the method of detecting the viral particle can detect the presence of at least about 3 viral particles.

26. The method of claim 17, wherein the antigen is a protein.

27. The method of claim 26, wherein the method of detecting the protein can detect the presence of at least about 15 ng of protein.

28. The method of claim 26, wherein the method of detecting the protein can detect the presence of at least about 5 ng of protein.

29. The method of claim 26, wherein the method of detecting the protein can detect the presence of at least about 0.01 ng of protein.

30. A method of killing bacteria comprising:
(a) obtaining a fluorescent nanoparticle conjugated to a substance capable of binding specifically to a bacteria to form a conjugated fluorescent nanoparticle;
(b) placing the conjugated fluorescent nanoparticle in a location where the bacteria is suspected to be; and
(c) binding the conjugated fluorescent nanoparticle to the bacteria, wherein the method of killing is not due to thermal activation.

31. A method of claim 30, further comprising a log kill of 0.5.

32. A method of killing bacteria comprising:
(a) obtaining a fluorescent nanoparticle comprising at least one terminal group capable of being used for conjugation;
(b) placing the fluorescent nanoparticle comprising at least one terminal group capable of being used for conjugation in a location where the bacteria is suspected to be; and
(c) binding the conjugated fluorescent nanoparticle to the bacteria, wherein the method of killing is not due to thermal activation.

33. A method of claim 32, further comprising a log kill of 0.14.

34. A method of killing bacteria comprising:
(a) obtaining a fluorescent nanoparticle conjugated to a substance capable of binding specifically to a bacteria to form a conjugated fluorescent nanoparticle;
(b) placing the conjugated fluorescent nanoparticle in a location where where the bacteria is suspected to be;
(c) binding the conjugated fluorescent nanoparticle to the bacteria; and
(d) exposing the location to microwaves.

35. A method of detecting two or more types of bacteria comprising:
(a) obtaining a first fluorescent nanoparticle conjugated to a substance capable of binding specifically to a bacteria to form a first conjugated fluorescent nanoparticle, wherein the fluorescent nanoparticle conjugated to a substance capable of binding specifically to a bacteria emits at one wavelength;
(b) obtaining a second fluorescent nanoparticle conjugated to a substance capable of binding specifically to a bacteria to form a second conjugated fluorescent nanoparticle, wherein the fluorescent nanoparticle conjugated to a substance capable of binding specifically to a bacteria emits at another wavelength;
(c) placing the first and second conjugated fluorescent nanoparticles in a location where the bacteria is suspected to be;
(d) exposing the location to a wavelength of light capable of exciting the first and second conjugated fluorescent nanoparticles;
(e) measuring fluorescence emission of the first and second conjugated fluorescent nanoparticles; and
(f) observing the wavelength of the measured fluorescence emission of step (e) in comparison with the wavelength of the fluorescence emission of the first and second conjugated fluorescent nanoparticles that have not been exposed to the bacteria wherein the first and second conjugated fluorescent nanoparticles exhibit lower emission wavelengths upon binding to the bacteria.

36. A composition for use in detection of bacteria comprising a fluorescent nanoparticle conjugated to a substance capable of binding specifically to a bacteria to form a conjugated fluorescent nanoparticle wherein the conjugated fluorescent nanoparticle exhibits a lower emission peak wavelength upon binding to the bacteria.

37. A composition for use in detection of bacteria comprising a fluorescent nanoparticle conjugated to a substance capable of binding specifically to an antigen to form a conjugated fluorescent nanoparticle wherein the conjugated fluorescent nanoparticle exhibits a lower emission peak wavelength upon binding to the antigen.

38. A composition for killing bacteria comprising a fluorescent nanoparticle conjugated to a substance capable of binding specifically to a bacteria to form a conjugated fluorescent nanoparticle wherein the conjugated fluorescent nanoparticle exhibits a lower emission peak wavelength upon binding to the bacteria and the killing is not due to thermal activation.

39. A composition for detecting two or more types of bacteria comprising a first and second fluorescent nanoparticle conjugated to substances capable of binding specifically to the two or more types of bacteria to form a first and second conjugated fluorescent nanoparticle wherein the first and second conjugated nanoparticles emit at different wavelengths and exhibit a lower emission peak wavelength upon binding to bacteria.

40. A composition for detecting two or more types of antigen comprising a first and second fluorescent nanoparticle conjugated to substances capable of binding specifically to the two or more types of antigen to form a first and second conjugated fluorescent nanoparticles wherein the first and second conjugated nanoparticles emit at different wavelengths and exhibit a lower emission peak wavelength upon binding to the two or more types of antigen.
